(19) United States
(12) Patent Application Publication
(10) Pub. No.: US 2016/0266086 A1
(43) Pub. Date: Sep. 15, 2016

(54) CEMENT INTEGRITY SENSORS AND METHODS OF MANUFACTURE AND USE THEREOF

(71) Applicant: MICROMEM APPLIED SENSOR TECHNOLOGIES INC., New York, NY (US)

(72) Inventors: Brian VON HERZEN, Minden, NV (US); Steven Van Fleet, Lagrangeville, NY (US)

(21) Appl. No.: 15/064,469

(22) Filed: Mar. 8, 2016

Related U.S. Application Data
(60) Provisional application No. 62/130,269, filed on Mar. 9, 2015.

(51) Int. Cl. G01N 33/38; G01N 25/00; G01N 29/04; G01N 3/40

(52) U.S. Cl. CPC G01N 33/383; G01N 3/40; G01N 25/00; G01N 29/04; G01N 2291/0232

(57) ABSTRACT

The invention encompasses systems and methods for detecting and/or monitoring the integrity and/or condition of cement, structures incorporating cement including, for example, highways, bridges, buildings, and wellbores using Nano-Electro-Mechanical System (NEMS)-based and/or Micro-Electro-Mechanical System (MEMS)-based data sensors. The disclosure further encompasses systems and methods of monitoring the integrity and performance of a structure and the surrounding formation of structure through the life of the structure using NEMS/MEMS-based data sensors.

### CROSS-REFERENCE TO RELATED APPLICATIONS

The present application claims benefit of U.S. Provisional Patent Application No. 62/130,269, filed Mar. 9, 2015, the disclosure of which is incorporated by reference herein in its entirety.

### FIELD OF THE INVENTION

The invention encompasses systems and methods for detecting and/or monitoring the integrity and/or condition of cement, structures incorporating cement including, for example, highways, bridges, buildings, and wellbores using Nano-Electro-Mechanical System (NEMS)-based and/or Micro-Electro-Mechanical System (MEMS)-based data sensors. The disclosure further encompasses systems and methods of monitoring the integrity and performance of a structure and the surrounding formation of a structure through the life of the structure using NEMS/MEMS-based data sensors.

### BACKGROUND OF THE INVENTION

Curing cement requires a lengthy and careful process to achieve maximum strength and hardness. Knowing when it is strong enough to use can save time, resources and complexity in the building process. Minute sensors can be harmlessly embedded in the cement to inform the maker of the cement's strength, hardness, and hydration, for example. Such sensors are also useful for monitoring the environmental conditions of the cement over the lifetime of the structure.

Smart sensing has a number of structural applications. In civil engineering, it can confirm cement and concrete integrity, monitor the curing process, and measure reliability. Factors that can affect cement integrity can include potential mechanical gaps in the installation, curing, geomechanical stress and strain, temperature, autogenous shrinkage, flowing fluids, pH, presence and concentration of particular ions (such as chloride, carbon dioxide or acidic conditions), carbonation, and microfracturing. Under adverse environmental conditions, particularly high pressure, the stress/strain situations can be intense enough to crack sensor materials. Hence hard casings are often required for devices to withstand such stresses.

A traditional method of measuring cement curing is the temperature curve over time. Curing is an exothermic process. Observing a temperature rise and maximum provides some information about the present stage of the curing process and completion, Such temperature-time history can estimate cement maturity through the curing process. When a large volume of cement is positioned, the thermodynamics of the curing process as well as its geometry can cause temperature gradients to occur. Data loggers, sometimes called “maturity meters,” are often used. One drawback is that typical equipment is vulnerable to corrosion and other environmental conditions, so it typically cannot remain on site for long periods. The heat method also provides a limited set of data as its focus is on hydration.

An ongoing need exists for improvements related to detecting and/or monitoring the integrity and/or condition of cement, and structures incorporating cement. Such needs may be meet b the novel and inventive systems and methods for use of NEMS/MEMS-based sensors in accordance with the various embodiments described herein.

### SUMMARY OF THE INVENTION

The invention general encompasses the use of sensors to determine the integrity of cement utilized in various structures including, for example, wellbores, bridges, and buildings.

In one embodiment, the invention encompasses a sensor comprising:
a temperature sensing element;
a pressure sensing element;
a stress/strain sensing element; and
an acoustic sensing element
wherein the sensor component is on the scale of about centimeters to about microns.

In another embodiment, the invention encompasses a cement monitoring composition comprising a plurality of wireless sensors, wherein each sensor comprises:
a sensor component comprising:
a temperature sensing element;
a pressure sensing element;
a stress/strain sensing element; and
an acoustic sensing element,
wherein the sensor component is on the scale of centimeters to about microns.

In certain embodiments, the sensor component comprises a polymer material.

In certain embodiments, the polymer material comprises a polymer film material.

In certain embodiments, the polymer film material comprises polyimide.

In certain embodiments, the sensor component comprises a ceramic material.

In certain embodiments, the ceramic material comprises a ceramic perovskite material,

In certain embodiments, the ceramic material is lead zirconium titanate.

In certain embodiments, the sensor component has a dielectric constant from about 200 to about 4000.

In certain embodiments, the sensor component comprises a piezoelectric material.

In certain embodiments, the temperature sensing element is a temperature diode.

In certain embodiments, the temperature sensing element is thermistor.

In certain embodiments, the pressure sensing element is a pressure sensitive ink.

In certain embodiments, the pressure sensing element is a pressure sensitive transducer.

In certain embodiments, the pressure sensing element comprises a passivation layer.

In certain embodiments, the stress/strain sensing element is a nanoparticle-based strain gauge.

In certain embodiments, the stress/strain sensing element is a foil strain gauge.

In certain embodiments, the stress/strain sensing element comprises an interdigitated transducer.

In certain embodiments, the cement monitoring composition further comprises one or more data collection components.

In certain embodiments, the data collection component provides energizing functions to the sensors and data telemetry relay functions to collect data from the sensors.

In certain embodiments, the sensors collect data from a wellbore and transmit data to the data collection components.

In certain embodiments, the data collection components relay data from the wellbore.

In certain embodiments, the data collection components are located on the outside of a wellbore.

In certain embodiments, the data collection components are located or inside of a wellbore.

Another embodiment encompasses a method of monitoring a cement comprising:
providing a plurality of wireless sensors in a cement, wherein each sensor comprises:
   a sensor component comprising:
   a temperature sensing element;
   a pressure sensing element;
   a stress/strain sensing element; and
an acoustic sensing element
adding the cement to a wellbore;
obtaining data from the sensors using a plurality of data collection components spaced along a length of the wellbore; and
transmitting the data obtained from the sensors from an interior of the wellbore to an exterior of the wellbore.

In certain embodiments, the sensor component is on the scale of centimeters to about microns.

In certain embodiments, the sensor component comprises a polymer material.

In certain embodiments, the polymer material comprises a polymer film material.

In certain embodiments, the polymer film material comprises polyimide.

In certain embodiments, the sensor component comprises a ceramic material.

In certain embodiments, the ceramic material comprises a ceramic perovskite material.

In certain embodiments, the ceramic material is lead zirconium titanate.

In certain embodiments, the sensor component has a dielectric constant from about 200 to about 4000.

In certain embodiments, the sensor component comprises a piezoelectric material.

In certain embodiments, the temperature sensing element is a temperature diode.

In certain embodiments, the temperature sensing element is thermistor.

In certain embodiments, the pressure sensing element is a pressure sensitive ink.

In certain embodiments, the pressure sensing element is a pressure sensitive transducer.

In certain embodiments, the pressure sensing element comprises a passivation layer.

In certain embodiments, the stress/strain sensing element is a nanoparticle-based strain gauge.

In certain embodiments, the stress/strain sensing element is a foil strain gauge.

In certain embodiments, the stress/strain sensing element comprises an interdigitated transducer.

In certain embodiments, the data collection component provides energizing functions to the sensors and data telemetry relay functions to collect data from the sensors.

In certain embodiments, the sensors collect data from a wellbore and transmit data to the data collection components.

In certain embodiments, the data collection components relay data from the wellbore.

In certain embodiments, the data collection components are located on the outside of a wellbore.

In certain embodiments, the data collection components are located on the inside of a wellbore.

### BRIEF DESCRIPTION OF THE FIGURES

FIG. 1 illustrates an illustrative embodiment of wireless data collection components (“hubs”) deployed outside of the casing to monitor cement integrity.

FIG. 2. illustrates an illustrative embodiment of wireless hubs deployed inside of the casing to monitor cement integrity.

FIG. 3 illustrates an illustrative remote sensors exchanging data wirelessly with hubs, the hubs in turn also communicating with a back-end data processing system at the surface of a wellbore.

FIG. 4 illustrates an illustrative diagram of a NEMS or MEMS sensor 400 comprising temperature sensing element 402, pressure sensing element 404, stress/strain sensing element 406, and acoustic sensing element 408.

### DETAILED DESCRIPTION OF THE INVENTION

The invention encompasses a system comprising a smart sensing cement that is capable of real-time, continuous monitoring of cement conditions, for example, over the lifetime of a subterranean well such as a hydrocarbon recovery well. In certain embodiments, this enables routine monitoring as well as critical monitoring during the cement curing process, critical monitoring during the drilling operation process, but also provides an archive of the history of the conditions inside the well over the construction period and over extended periods of time. In certain embodiments, the invention encompasses extensive logging data that can be useful in determining causes of any difficulties or problems or engineering analysis.

The methods and compositions are generally designed to assess cement characterization and integrity over time. The compositions and methods comprise detecting and/or monitoring the integrity of cement using NEMS-based and/or MEMS-based data sensors. In certain embodiments, the compositions and methods comprise monitoring the integrity and performance of cement compositions over the life of the cement using NEMS-based and/or MEMS-based sensors. The performance may be monitored, for example, by changes, for example, in various parameters, including, but not limited to, geomechanical stress and strain, temperature, autogenous shrinkage, flowing fluids, pH, presence and concentration of particular ions (such as, for example, carbonate, chloride, sodium, and potassium ions or acidic conditions), the presence of ammonia or nitrate, carbonation, microfracturing, and moisture content of the cement.

In certain embodiments, the methods and compositions comprise the use of a plurality of embeddable sensors capable of detecting parameters in a cement composition, for example, a wellbore sealant such as cement. In certain embodiments, the methods and compositions provide for evaluation of cement during mixing, placement, and/or curing of the cement. In another embodiment, the methods and compositions are used for cement evaluation from placement and curing throughout its useful service life, and where applicable to a period of deterioration and repair. In embodiments, methods are disclosed for determining the location of cement within a wellbore, such as for determining the location of a cement slurry during primary cementing of a wellbore. Additional embodiments and methods for employing NEMS-based or MEMS-based sensors are described herein.

In other embodiments, the NEMS or MEMS sensors are contained within a cement composition placed substantially within the annular space between a casing and the wellbore wall. In certain embodiments, substantially all of the NEMS or MEMS sensors are located within or in close proximity to the annular space. In an embodiment, the cement comprising the NEMS or MEMS sensors (and thus likewise the MEMS sensors) does not substantially penetrate, migrate, or travel into the formation from the wellbore. In an alternative embodiment, substantially all of the NEMS or MEMS sensors are located within, adjacent to, or in close proximity to the wellbore, for example less than or equal to about 1 foot, 3 feet, 5 feet, or 10 feet from the wellbore. Such adjacent or close proximity positioning of the sensors with respect to the wellbore is in contrast to placing NEMS or MEMS sensors in a fluid that is pumped into the formation in large volumes and substantially penetrates, migrates, or travels into or through the formation, for example as occurs with a fracturing fluid or a flooding fluid. Thus, in embodiments, the NEMS or MEMS sensors are placed proximate or adjacent to the wellbore (in contrast to the formation at large), and provide information relevant to the wellbore itself and compositions (e.g., sealants) used therein (again in contrast to the formation or a producing zone at large).

Examples of cements useful in the composition and methods of the invention include cementitious and non-cementitious sealants both of which are well known in the art. In embodiments, non-cementitious sealants comprise resin-based systems, latex-based systems, or combinations thereof. In embodiments, the sealant comprises a cement slurry with styrene-butadiene latex (e.g., as disclosed in U.S. Pat. No. 5,588,488 incorporated by reference herein in its entirety). Sealants may be utilized in setting expandable casing, which is further described herein below. In other embodiments, the sealant is a cement utilized for primary or secondary wellbore cementing operations, as discussed further herein.

in embodiments, the cement comprises a hydraulic cement that sets and hardens by reaction with water. Examples of hydraulic cements include but are not limited to Portland cements (e.g., classes A, B, C, G, and H Portland cements), pozzolana cements, gypsum cements, phosphate cements, high alumina content cements, silica cements, high alkalinity cements, shale cements, acid/base cements, magnesia cements, fly ash cement, zeolite cement systems, cement kiln dust cement systems, slag cements, micro-fine cement, metakaolin, and combinations thereof. Examples of sealants are disclosed in U.S. Pat. Nos. 6,457,524; 7,077,203; and 7,174,962, each of which is incorporated herein by reference in its entirety. In an embodiment, the sealant comprises a sorel cement composition, which typically comprises magnesium oxide and a chloride or phosphate salt which together form for example magnesium oxychloride. Examples of magnesium oxychloride sealants are disclosed in U.S. Pat. Nos. 6,664,215 and 7,044,222, each of which is incorporated herein by reference in its entirety.

The wellbore composition may include a sufficient amount of water to form a pumpable slurry. The water may be fresh water or salt water (e.g., an unsaturated aqueous salt solution or a saturated aqueous salt solution such as brine or seawater). In embodiments, the cement slurry may be a lightweight cement slurry containing foam (e.g., foamed cement) and/or hollow beads/microspheres. In an embodiment, the NEMS or MEMS sensors are incorporated into or attached to all or a portion of the hollow microspheres. Thus, the sensors may be dispersed within the cement along with the microspheres. Examples of sealants containing microspheres are disclosed in U.S. Pat. Nos. 4,234,344; 6,457,524; and 7,174,962, each of which is incorporated herein by reference in its entirety. In an embodiment, the NEMS or MEMS sensors are incorporated into a foamed cement such as those described in more detail in U.S. Pat. Nos. 6,063,738; 6,367,550; 6,547,871; and 7,174,962, each of which is incorporated by reference herein in its entirety.

In some embodiments, additives may be included in the cement composition for improving or changing the properties thereof Examples of such additives include but are not limited to accelerators, set retarders, defoamers, fluid loss agents, weighting materials, dispersants, density-reducing agents, formation conditioning agents, lost circulation materials, thixotropic agents, suspension aids, or combinations thereof. Other mechanical property modifying additives, for example, fibers, polymers, resins, latexes, and the like can be added to further modify the mechanical properties. These additives may be included singularly or in combination. Methods for introducing these additives and their effective amounts are known to one of ordinary skill in the art.

In certain embodiments, the NEMS or MEMS sensors are contained within a cement, and can be provided, along with a wellbore composition that when placed downhole under suitable conditions induces fractures within the subterranean formation. Hydrocarbon-producing wells often are stimulated by hydraulic fracturing operations, wherein a fracturing fluid may be introduced into a portion of a subterranean formation penetrated by a wellbore at a hydraulic pressure sufficient to create, enhance, and/or extend at least one fracture therein. Stimulating or treating the wellbore in such ways increases hydrocarbon production from the well. In such embodiments, the NEMS or MEMS sensors provide information as to the location and/or condition of cement, as well as potentially the fluid and/or fracture during and/or after treatment. In an embodiment, at least a portion of the NEMS or MEMS sensors are associated with a fracturing fluid and may provide information as to the condition and/or location of the fluid. Fracturing fluids often contain proppants that are deposited within the formation upon placement of the fracturing fluid therein, and in an embodiment a fracturing fluid contains one or more proppants and can further contain one or more NEMS or MEMS sensors.

In embodiments, the NEMS or MEMS sensors are contained in a cement that is also provided with a wellbore composition (e.g., gravel pack fluid) which is employed in a gravel packing treatment, and the NEMS or MEMS may provide information as to the condition and/or location of the wellbore composition during and/or after the gravel packing treatment. Gravel packing treatments are used, inter alia, to reduce the migration of unconsolidated formation particulates into the wellbore. In gravel packing operations, particulates, referred to as gravel, are carried to a wellbore in a subterranean producing zone by a servicing fluid known as carrier fluid. That is, the particulates are suspended in a carrier fluid, which may be viscosified, and the carrier fluid is pumped into a wellbore in which the gravel pack is to be placed. As the particulates are placed in the zone, the carrier fluid leaks off into the subterranean zone and/or is returned to the surface. The resultant gravel pack acts as a filter to separate formation solids from produced fluids while permitting the produced fluids to flow into and through the wellbore. When installing the gravel pack, the gravel is carried to the formation in the form of a slurry by mixing the gravel with a viscosified carrier fluid. Such gravel packs may be used to stabilize a formation while causing minimal impairment to well productivity. The gravel, inter alia, acts to prevent the particulates from occluding the screen or migrating with the produced fluids, and the screen, inter alia, acts to prevent the gravel from entering the wellbore. In an embodiment, the wellbore servicing composition (e.g., gravel pack fluid) comprises a carrier fluid, gravel and one or more NEMS or MEMS sensors. In an embodiment, at least a portion of the NEMS or MEMS remain associated with the gravel deposited within the wellbore and/or formation (e.g., a gravel pack/bed) and may provide information as to the condition (e.g., thickness, density, settling, stratification, integrity, etc.) and/or location of the gravel pack/bed.

In various embodiments, the NEMS/MEMS sensors may provide information as to a location, flow path/profile, volume, density, temperature, pressure, stress-strain or a combination thereof of a cement, a sealant composition, a drilling fluid, a fracturing fluid, a gravel pack fluid, or other wellbore servicing fluid in real time such that the effectiveness of such service may be monitored and/or adjusted during performance of the service to improve the result of same. Accordingly, the NEMS or MEMS sensors may aid in the initial performance of the wellbore service additionally or alternatively to providing a means for monitoring a wellbore condition or performance of the service over a period of time (e.g., over a servicing interval and/or over the life of the well). For example, the one or more NEMS or MEMS sensors may be used in monitoring a gas or a liquid produced from the subterranean formation. NEMS or MEMS sensors present in the wellbore and/or formation may be used to provide information as to the condition (e.g., temperature, pressure, flow rate, stress-strain, composition, etc.) and/or location of a gas or liquid produced from the subterranean formation. In an embodiment, the NEMS or MEMS sensors provide information regarding the composition of a produced gas or liquid. For example, the NEMS or MEMS sensors may be used to monitor an amount of water produced in a hydrocarbon producing well (e.g., amount of water present in hydrocarbon gas or liquid), an amount of undesirable components or contaminants in a produced gas or liquid (e.g., sulky, carbon dioxide, hydrogen sulfide, etc. present in hydrocarbon gas or liquid), or a combination thereof

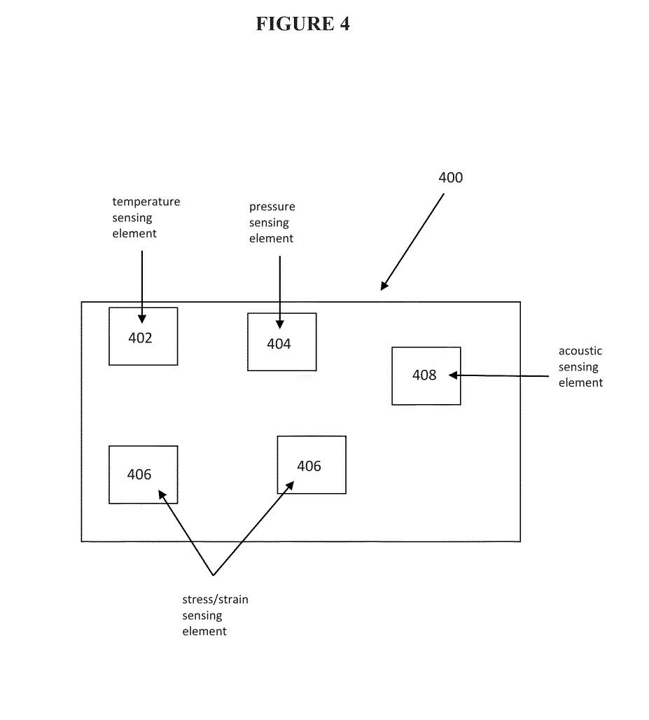

In embodiments, as shown in FIG. 4, provided herein are sensor components 400 (shown as a genetic diagram illustrating the several components). Suitably sensor component, which is a NEMS or MEMS sensor, comprises temperature sensing element 402, pressure sensing element 404, stress/strain sensing element 406 and acoustic sensing element 408. As described throughout, sensor component 400 is suitable on the scale of about centimeters to about microns. The several components (402-408) of sensor component 400 can be electrically integrated or connected using methods known in the art, including for example roll-to-roll processing as described herein.

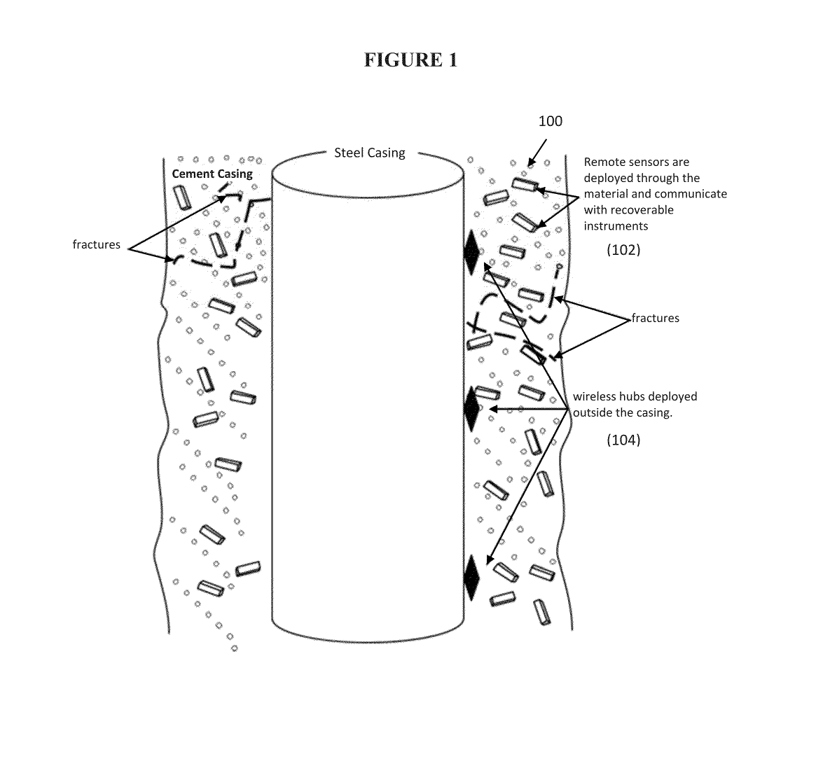

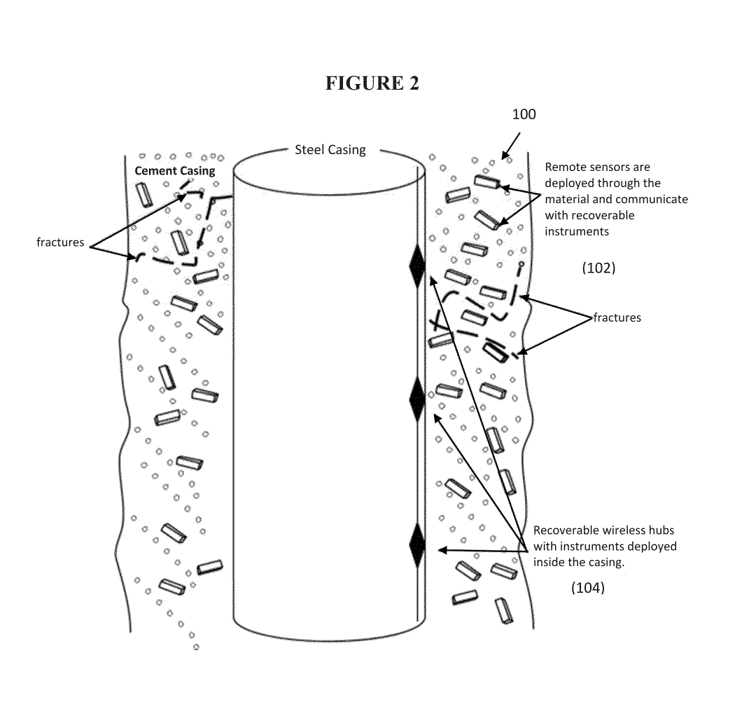

In further embodiments, a cement monitoring composition 100, as shown in FIGS. 1 and 2 is provided, comprising a plurality of wireless sensors 102, wherein each sensor comprises a sensor component 400 comprising a temperature sensing element 402, a pressure sensing element 404, a stress/strain sensing element 406, and an acoustic sensing element 408. As described herein, suitably sensor component is on the scale of centimeters to about microns.

In embodiments, the sensor component comprises a polymer material, including various polymer materials described herein, for example, a polymer film material. Such polymer film materials can comprise polyimide.

In additional embodiments, the sensor component comprises a ceramic material, such as, but not limited to a ceramic perovskite material or a lead zirconium titanate. 1741 Suitably, the sensor component has a dielectric constant from about 200 to about 4000.

In further embodiments, the sensor component comprises a piezoelectric material.

As described herein, the temperature sensing element is suitably a temperature diode, or can be a thermistor.

In embodiments, pressure sensing element is a pressure sensitive ink, or can be a pressure sensitive transducer, and the pressure sensing element can suitably comprise a passivation layer.

Suitably, the stress/strain sensing element is a nanoparticle-based strain gauge, or can be a foil strain gauge. In embodiments, the stress/strain sensing element comprises an interdigitated transducer.

In embodiments, the cement monitoring compositions described herein suitably further comprising one or more data collection components 104 as shown in FIGS. 1 and Such data collection components can provide energizing functions to the sensors and data telemetry relay functions to collect data from the sensors. Suitably, the sensors collect data from a wellbore and transmit data to the data collection components. Suitably, data collection components relay data from the wellbore.

As shown in FIG. 1, in embodiments, data collection components 104 (also called hubs throughout) are located on the outside of a wellbore. In other embodiments, as shown in FIG. 2, data collection components 104 are located on the inside of a wellbore.

In various embodiments, the NEMS or MEMS sensors sense one or more parameters of the cement within the wellbore. In an embodiment, the parameter is temperature. Alternatively, the parameter is pH. Alternatively, the parameter is moisture content. Still alternatively, the parameter may be ion concentration (e.g., chloride, sodium, and/or potassium ions). The NEMS/MEMS sensors may also sense cement characteristic data such as stress, strain, or combinations thereof.

In addition or in the alternative, a NEMS or MEMS sensor incorporated within one or more of the wellbore compositions disclosed herein (including cement) may provide information that allows a condition (e.g., thickness, density, volume, settling, stratification, etc.) and/or location of the composition within the subterranean formation to be detected. In embodiments, multiple different wellbore compositions can be prepared and provided together, or separately, each comprising sensor components depending on the desired measurement or information to be gathered.

Generally, a communication distance between NEMS/MEMS sensors varies with a size and/or mass of the NEMS/MEMS sensors. However, an ability to suspend the NEMS/MEMS sensors in a wellbore composition and keep the NEMS/MEMS sensors suspended in the wellbore composition for a long period of time, which may be important for measuring various parameters of a wellbore composition throughout a volume of the wellbore composition, generally varies inversely with the size of the NEMS/MEMS sensors. Therefore, sensor communication distance requirements may have to be adjusted in view of sensor suspendability requirements. In addition, a communication frequency of a NEMS/MEMS sensor generally varies with the size and/or mass of the NEMS/MEMS sensor.

In embodiments, the sensors are ultra-small, e.g., 3 mm² such that they are pumpable in a cement or other wellbore composition, In embodiments, the sensor components (also called sensors, or NEMS/MEMS sensors throughout) are approximately 0.01 mm² to 1 mm² alternatively 1 mm² to 3 mm², alternatively 3 mm² to 5 mm², or alternatively 5 mm² to 10 mm². In embodiments, the data sensors are capable of providing data throughout the cement service life. In embodiments, the data sensors are capable of providing data for 1-10 years, for 1-20 years, for 1-30 years, for 1-40 years, for 1-50 years, for 1-60 years, for 1-70 years, for 1-80 years, for 1-90 years, or up to 100 years.

In an embodiment, the wellbore composition (e.g., cement) comprises an amount of sensor components effective to measure one or more desired parameters. In various embodiments, the wellbore composition comprises an effective amount of sensor components such that sensed readings may be obtained at intervals of about 1 foot, alternatively about 6 inches, or alternatively about 1 inch, along the portion of the wellbore containing the sensor components. In an embodiment, the sensor components may be present in the cement or other wellbore composition in an amount of from about 0.001 to about 10 weight percent. Alternatively, the sensor components may be present in the wellbore composition in an amount of from about 0.01 to about 5 weight percent. In embodiments, the sensors may have dimensions (e.g., diameters or other dimensions) that range from nanoscale, e.g., about 1 to 1000 nm (e.g., NEMS), to a micrometer range, e.g., about 1 to 1000 μm (e.g., MEMS), or alternatively any size from about 1 nm to about 1 mm. In embodiments, the sensor components sensors may be present in the wellbore composition in an amount of from about 5 volume percent to about 30 volume percent.

In various embodiments, the size and/or amount of sensor components present in a wellbore composition (e.g., the sensor loading or concentration) may be selected such that the resultant wellbore servicing composition (such as cement) is readily pumpable without damaging the sensors and/or without having the sensors undesirably settle out (e.g., screen out) in the pumping equipment (e.g., pumps, conduits, tanks, etc.) and/or upon placement in the wellbore. Also, the concentration/loading of the sensors within the the wellbore servicing fluid may be selected to provide a sufficient average distance between sensors to allow for networking of the sensors (e.g., daisy-chaining) in embodiments using such networks, as described in more detail herein. For example, such distance may be a percentage of the average communication distance for a given sensor type. By way of example, a given sensor having a 2 inch communication range in a given wellbore composition should be loaded into the wellbore composition in an amount that the average distance between sensors in less than 2 inches (e.g., less than 1.9, 1.8, 1.7, 1.6, 1.5, 1.4, 1.3, 1.2, 1.1, 1.0, etc. inches). The size of sensors and the amount may be selected so that they are stable, do not float or sink, in the well treating fluid. The size of the sensor could range from nano size to microns. In some embodiments, the sensors may be nanoelectromechanical systems (NEMS), MEMS, or combinations thereof. Unless otherwise indicated herein, it should be understood that any suitable micro and/or nano sized sensors or combinations thereof may be employed. The embodiments disclosed herein should not otherwise be limited by the specific type of micro and/or nano sensor employed unless otherwise indicated or prescribed by the functional requirements thereof, and specifically NEMS may be used in addition to or in lieu of MEMS sensors in the various embodiments disclosed herein.

Secondary cementing within a wellbore may be carried out subsequent to primary cementing operations. A common example of secondary cementing is squeeze cementing wherein a sealant such as a cement composition is forced under pressure into one or more permeable zones within the wellbore to seal such zones. Examples of such permeable zones include fissures, cracks, fractures, streaks, flow channels, voids, high permeability streaks, annular voids, or combinations thereof. The permeable zones may be present in the cement column residing in the annulus, a wall of the conduit in the wellbore, a microannulus between the cement column and the subterranean formation, and/or a microannulus between the cement column and the conduit. The sealant (e.g., secondary cement composition) sets within the permeable zones, thereby forming a hard mass to plug those zones and prevent fluid from passing therethrough (i.e., prevents communication of fluids between the wellbore and the formation via the permeable zone). Various procedures that may be followed to use a sealant composition in a wellbore are described in U.S. Pat. No. 5,346,012, which is incorporated by reference herein in its entirety. In various embodiments, a sealant composition comprising MEMS sensors is used to repair holes, channels, voids, and microannuli in casing, cement sheath, gravel packs, and the like as described in U.S. Pat. Nos. 5,121,795; 5,123,487; and 5,127,473, each of which is incorporated by reference herein in its entirety.

In embodiments, the method of the present disclosure may be employed in a secondary cementing operation. In these embodiments, sensor components are mixed with a sealant composition (e.g., a secondary cement slurry) and subsequent or during positioning and hardening of the cement, the sensors are interrogated to monitor the performance of the secondary cement in an analogous manner to the incorporation and monitoring of the data sensors in primary cementing methods disclosed herein. For example, the MEMS sensors may be used to verify the location of the secondary sealant, one or more properties of the secondary sealant, that the secondary sealant is functioning properly and/or to monitor its long-term integrity.

In embodiments, the methods of the present disclosure are utilized for monitoring cementitious sealants (e.g., hydraulic cement), non-cementitious (e.g., polymer, latex or resin systems), or combinations thereof, which may be used in primary, secondary, or other sealing applications. For example, expandable tubulars such as pipe, pipe string, casing, liner, or the like are often sealed in a subterranean formation. The expandable tubular (e.g., casing) is placed in the wellbore, a sealing composition is placed into the wellbore, the expandable tubular is expanded, and the sealing composition is allowed to set in the wellbore. For example, after expandable casing is placed downhole, a mandrel may be run through the casing to expand the casing diametrically, with expansions up to 25% possible. The expandable tubular may be placed in the wellbore before or after placing the sealing composition in the wellbore. The expandable tubular may be expanded before, during, or after the set of the sealing composition. When the tubular is expanded during or after the set of the sealing composition, resilient compositions will remain competent due to their elasticity and compressibility. Additional tubulars may be used to extend the wellbore into the subterranean formation below the first tubular as is known to those of skill in the art. Sealant compositions and methods of using the compositions with expandable tubulars are disclosed in U.S. Pat. Nos. 6,722,433 and 7,040,404 and U.S. Pat. Pub. No. 2004/0167248, each of which is incorporated by reference herein in its entirety. In expandable tubular embodiments, the sealants may comprise compressible hydraulic cement compositions and/or non-cementitious compositions.

Compressible hydraulic cement compositions have been developed which remain competent (continue to support and seal the pipe) when compressed, and such compositions may comprise sensor components. The sealant composition is placed in the annulus between the wellbore and the pipe or pipe string, the sealant is allowed to harden into an impermeable mass, and thereafter, the expandable pipe or pipe string is expanded whereby the hardened sealant composition is compressed, In embodiments, the compressible foamed sealant composition comprises a hydraulic cement, a rubber latex, a rubber latex stabilizer, a gas and a mixture of foaming and foam stabilizing surfactants. Suitable hydraulic cements include, but are not limited to, Portland cement and calcium aluminate cement.

Often, non-cementitious resilient sealants with comparable strength to cement, but greater elasticity and compressibility, are required for cementing expandable casing. In embodiments, these sealants comprise polymeric sealing compositions, and such compositions may comprise MEMS sensors, In an embodiment, the sealants composition comprises a polymer and a metal containing compound. In embodiments, the polymer comprises copolymers, terpolymers, and interpolymers, The metal-containing compounds may comprise zinc, tin, iron, selenium magnesium, chromium, or cadmium, The compounds may be in the form of an oxide, carboxylic acid salt, a complex with dithiocarbamate ligand, or a complex with mercaptobenzothiazole ligand. In embodiments, the sealant comprises a mixture of latex, dithio carbamate, zinc oxide, and sulfur.

In embodiments, the methods of the present disclosure comprise adding data sensors to a sealant to be used behind expandable casing to monitor the integrity of the sealant upon expansion of the casing and during the service life of the sealant. In this embodiment, the sensors may comprise MEMS sensors capable of measuring, for example, moisture and/or temperature change. If the sealant develops cracks, water influx may thus be detected via moisture and/or temperature indication.

In an embodiment, the sensor components are added to one or more wellbore servicing compositions used or placed downhole in drilling or completing a monodiameter wellbore as disclosed in U.S. Pat. No. 7,066,284 and U.S. Pat. Pub. No. 2005/0241855, each of which is incorporated by reference herein in its entirety. In an embodiment, the sensor components are included in a chemical casing composition used in a monodiameter wellbore. In another embodiment, the sensor components are included in compositions (e.g., sealants) used to place expandable casing or tubulars in a monodiameter wellbore. Examples of chemical casings are disclosed in U.S. Pat. Nos. 6,702,044; 6,823,940; and 6,848,519, each of which is incorporated herein by reference in its entirety.

In one embodiment, the sensor components are used to gather data, e.g., cement data, and monitor the long-term integrity of the wellbore composition, e.g., cement composition, placed in a wellbore, for example a wellbore for the recovery of natural resources such as water or hydrocarbons or an injection well for disposal or storage. In an embodiment, data/information gathered and/or derived from sensor components in a downhole wellbore composition e.g., cement composition, comprises at least a portion of the input and/or output to into one or more calculators, simulations, or models used to predict, select, and/or monitor the performance of wellbore compositions e.g., sealant compositions, over the life of a well. Such models and simulators may be used to select a wellbore composition, e.g., cement composition, comprising sensor components for use in a wellbore. After placement in the wellbore, the sensor components may provide data that can be used to refine, recalibrate, or correct the models and simulators. Furthermore, the sensor components can be used to monitor and record the downhole conditions that the composition, e.g., cement, is subjected to, and composition, e.g., cement, performance may be correlated to such long term data to provide an indication of problems or the potential for problems in the same or different wellbores. In various embodiments, data gathered from MEMS sensors is used to select a wellbore composition, e.g., cement composition, or otherwise evaluate or monitor such sealants, as disclosed in U.S. Pat. Nos. 6,697,738; 6,922,637; and 7,133,778, each of which is incorporated by reference herein in its entirety.

In an embodiment, the compositions and methodologies of this disclosure are employed in an operating environment that generally comprises a wellbore that penetrates a subterranean formation for the purpose of recovering hydrocarbons, storing hydrocarbons, injection of carbon dioxide, storage of carbon dioxide, disposal of carbon dioxide, and the like, and the sensor components located downhole (e.g., within the wellbore and/or surrounding formation) may provide information as to a condition and/or location of the composition and/or the subterranean formation. For example, the sensor components may provide information as to a location, flow path/profile, volume, density, temperature, pressure, or a combination thereof of a hydrocarbon (e.g., natural gas stored in a salt dome) or carbon dioxide placed in a subterranean formation such that effectiveness of the placement may be monitored and evaluated, for example detecting leaks, determining remaining storage capacity in the formation, etc. In some embodiments, the compositions of this disclosure are employed in an enhanced oil recovery operation wherein a wellbore that penetrates a subterranean formation may be subjected to the injection of gases (e.g., carbon dioxide) so as to improve hydrocarbon recovery from said wellbore, and the sensor components may provide information as to a condition and/or location of the composition and/or the subterranean formation, For example, the sensor components may provide information as to a location, flow path/profile, volume, density, temperature, pressure, or a combination thereof of carbon dioxide used in a carbon dioxide flooding enhanced oil recovery operation in real time such that the effectiveness of such operation may be monitored and/or adjusted in real time during performance of the operation to improve the result of same.

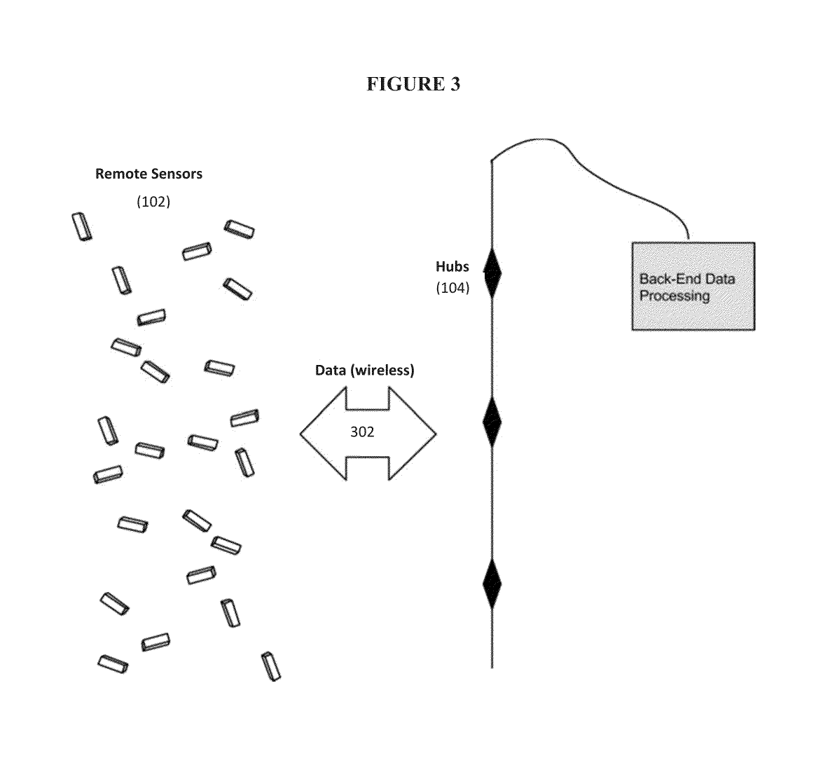

In embodiments, methods of monitoring a cement are provided. Such methods suitably comprise providing a plurality of wireless sensors in a cement, wherein each sensor comprises a sensor component (e.g., 400 of FIG. 4). The sensor component suitably comprises a temperature sensing element, a pressure sensing element, a stress/strain sensing element, and an acoustic sensing element. The methods further comprise adding the cement to a wellbore, obtaining data from the sensors using a plurality of data collection components spaced along a length of the wellbore, and transmitting the data obtained from the sensors from an interior of the wellbore to an exterior of the wellbore (302). See FIGS. 1-3.

As described herein, the sensor component is suitably on the scale of centimeters to about microns and can comprise a polymer material. Suitably, the polymer material is a polymer film material, such as polyimide. In other embodiments, the sensor component comprises a ceramic material, such as a ceramic perovskite material or lead zirconium titanate.

In embodiments, the sensor component has a dielectric constant from about 200 to about 4000. Suitably, the sensor component comprises a piezoelectric material.

In embodiments, the temperature sensing element is a temperature diode, or can be a thermistor.

In exemplary embodiments, the pressure sensing element is a pressure sensitive ink, or can be a pressure sensitive transducer, or can comprise a passivation layer.

Suitably, the stress/strain sensing element is a nanoparticle-based strain gauge. In other embodiments, the stress/strain sensing element is a foil strain gauge. In still further embodiments, the stress/strain sensing element comprises an interdigitated transducer.

As described throughout, the data collection component are suitably able to provide energizing functions to the sensors and data telemetry relay functions to collect data from the sensors. In embodiments, the sensors collect data from the cement and transmit data to the data collection components. Suitably, the data collection components relay data from the wellbore. The data collection components can be located on the outside of a wellbore, or in other embodiments, can be located on the inside of a wellbore.

### Sensors of the Invention

The various sensing elements (temperature, pressure, stress/strain and/or acoustic) are commercially available. In embodiments, the various sensing elements can be deposited directly onto a polymer film, such as a polyimide or Kapton film. A surface mount device can be utilized to directly connect to the Kapton printed circuit or it can be coupled. Various thermistor technologies provide resolution into the milli-degrees or fractions of a milli-degree. In embodiments, the various sensing elements can be printed onto a polymer, including for example Kapton or Mylar, using a roll-process so prepare the sensor components as printed electronic sensors.

For pressure sensors, the sensor components suitably utilize pressure-sensitive inks. There are a number of examples of pressure-sensitive materials that produce an electronic signal in response to an applied pressure which can be utilized in the embodiments described herein. With a suitable passivation layer that would chemically isolate and passivate the printed electronic circuit from the materials that may be present in and around a cement, there is less of a need for a pressure casing per se and a complete isolation with a suitable encapsulating materials is in general not necessary. In exemplary embodiments, silicon nitride can be used as a passivation material.

Additional passivation materials include various glasses and ceramics, and polymers as well. At moderate temperatures, polyimide can be considered to be an encapsulant or other passivation material in certain contexts. From that perspective, a variety of materials can be utilized for passivation and still achieve the objectives. In embodiments, the pressure and temperature sensing benefit from these encapsulants, from these passivation layers that would prolong their life while at the same time enabling the temperature and pressure sensing to be performed.

In embodiments, a strain gauge can be a traditional printed material, A zigzag pattern can be printed on a polymer, including a Kapton polyimide type of polymer. As the polymer is stretched or twisted, a difference in resistance or electronic signal results according to the directional strain that is placed upon the strain gauge. This miniaturizes the strain gauge further and embeds it in a wireless sensor infrastructure that becomes a bulk material that is actually mixed into the cement and provides very useful strain information during curing and for the life of the cement afterwards.

It is possible to determine the orientation of any particular stress/strain sensor using for example the gravitational field to the earth for a Z direction or vertical direction. Also the electromagnetic field of the earth can provide some indication of orientation: north, south, east, west. With that combination, even for a randomly oriented sensor that is embedded in the cement, with the combination of accelerometer, gravity sensors and these electromagnetic sensors measuring the field of the earth for example, an orientation of the sensor can be obtained. Thus if a strain gauge is responsive to strain in a particular direction, that direction can be measured by the sensor and incorporate it into the data analysis to determine the directional strain and stress strain relationships of cement throughout. In that way, it's possible to deliver an oriented holistic view of the cement stress strain in each dimensions and each stress-strain coordinates.

With regard to acoustic sensing and telemetry, up to a triple use of a piezo electric, ceramic or polymer material can be utilized. Piezo polymers and ceramics can be chemically vapor deposited, CVD, or can be applied using a wet or dried process to a film, and thus can comprise a thin film solution to acoustic energy scavenging, acoustic sensing and acoustic telemetry.

The sensitive films described herein suitably have this triple function. In that they are able to scavenge energy, listen for events (including flow, or other signals). They can record those signals and store them on the sensor. Ultimately they analyze those signals and transmit important results acoustically, suitably to the very same piezo transducer to the hub for transmission to the surface, relaying to the surface.

### Data Collection Components

Data collection components or hubs are suitably tuned in relation to remote sensor resonance. Remote sensors can respond with chirps that can be sent centered on different frequencies and/or at different times. Frequency bands are suitably chosen to minimize noise such as that associated with the flow of hydrocarbons through a separate tube in the borehole or other sources of noise. Collision detection methods may be implemented, for example using an exponential backoff algorithm when many remote sensors chirp at the same time. This is the same algorithm used in Ethernet packet signaling to avoid collisions, only here the medium is acoustic rather than electrical.

Hubs can pulse in at least three modes. The transponder mode is an interrogation mode, sending a short wake-up pulse to which the remote sensor responds with a chirp (ping or data packet). In the free-run mode, hubs send continuous wake-up pulses to which the remote sensor(s) respond by sending out multiple, spread-spectrum chirps (ping or data packet). The free-run mode stops when the hub no longer sends pulses and, therefore, the remote sensor stops chirping. In a phased-array mode, multiple hubs work together to target energy and waves to one or more remote sensors. Energy storage allows hubs to be left in place for permanent monitoring over long times.

Hubs inside or outside the well act as stimulus and/or response agents, querying and receiving responses from sensors. Hubs package the stimulus and/or response agents and are tolerant of oil or other fluids around them. A string of retrievable hubs containing transducers and/or other instruments is suitably deployed inside the well. The hubs can obtain data from remote sensor swarms as needed. At least three hubs in an array are suitably deployed above and below remote sensors of interest. Acoustic array receivers can fine-tune the location of the sensors, making it easier to achieve centimeter-scale resolution in many cases. Hubs function to provide power to the remote sensors, wake them from their quiet state and receive responses from the remote sensors. A ring of hubs can be deployed at one position along the borehole pipe, easily providing azimuthal position of the remote sensors. Hubs are placed on or near the pipe wall to minimize interference. Hubs can also match transducer to steel casing impedance by coupling firmly to the wall. In one embodiment with deployment inside the casing, expansion using a mechanism or balloon-type device pushes the hub against the casing. In another embodiment inside the casing, a biaxial braid is used with the string of hubs whose wider diameter when compressed pushes the hubs against the casing wall. Many stents in medical use have this type of expansion capability. Such techniques may be borrowed from arteriosclerotic medical fields.

Hubs can be deployed inside and outside the casing. They may also attach to the casing and can attach in different ways such as using mechanical structures, springs or transducers inside the casing or attaching to the outside of the casing with epoxy in a manner analogous to the way barnacles attach to hulls of ships.

In embodiments, the sensors comprise passive (remain unpowered when not being interrogated) sensors energized by energy radiated from a data interrogation tool. The data interrogation tool may comprise an energy transceiver sending energy (e.g., radio waves) to and receiving signals from the sensors and a processor processing the received signals. The data interrogation tool may further comprise a memory component, a communications component, or both. The memory component may store raw and/or processed data received from the sensors, and the communications component may transmit raw data to the processor and/or transmit processed data to another receiver, for example located at the surface. The tool components (e.g., transceiver, processor, memory component, and communications component) are coupled together and in signal communication with each other.

In an embodiment, one or more of the data collection components may be integrated into a tool or unit that is temporarily or permanently placed downhole (e.g., a downhole module), for example prior to, concurrent with, and/or subsequent to placement of the sensors in the wellbore. In an embodiment, a removable downhole module comprises a transceiver and a memory component, and the downhole module is placed into the wellbore, reads data from the sensors, stores the data in the memory component, is removed from the wellbore, and the raw data is accessed. Alternatively, the removable downhole module may have a processor to process and store data in the memory component, which is subsequently accessed at the surface when the tool is removed from the wellbore. Alternatively, the removable downhole module may have a communications component to transmit raw data to a processor and/or transmit processed data to another receiver, for example located at the surface. The communications component may communicate via wired or wireless communications. For example, the downhole component may communicate with a component or other node on the surface via a network of MEMS sensors, or cable or other communications/telemetry device such as a radio frequency, electromagnetic telemetry device or an acoustic telemetry device. The removable downhole component may be intermittently positioned downhole via any suitable conveyance, for example wire-line, coiled tubing, straight tubing, gravity, pumping, etc., to monitor conditions at various times during the life of the well.

Wireless power scavenging permits smart sensors to operate for extended periods without having to have a sustained internal power source. Wireless telemetry also enables measurements to be transmitted without wires. These two approaches enable remote sensing of cement curing and environmental conditions potentially for the life of the cement structure.

In embodiments, the data collection tool comprises a permanent or semi-permanent downhole component that remains downhole for extended periods of time. For example, a semi-permanent downhole module may be retrieved and data downloaded once every few months or years. Alternatively, a permanent downhole module may remain in the well throughout the service life of well. In an embodiment, a permanent or semi-permanent downhole module comprises a transceiver and a memory component, and the downhole module is placed into the wellbore, reads data from the sensors, optionally stores the data in the memory component, and transmits the read and optionally stored data to the surface. Alternatively, the permanent or semi-permanent downhole module may have a processor to process data into processed data, which may be stored in memory and/or transmit to the surface. The permanent or semi-permanent downhole module may have a communications component to transmit raw data to a processor and/or transmit processed data to another receiver, for example located at the surface. The communications component may communicate via wired or wireless communications. For example, the downhole component may communicate with a component or other node on the surface via a network of sensors, or a cable or other communications/telemetry device such as a radio frequency, electromagnetic telemetry device or an acoustic telemetry device.

In embodiments, the data interrogation tool comprises an RF energy source incorporated into its internal circuitry and the data sensors are passively energized using an RF antenna, which picks up energy from the RF energy source. In an embodiment, the data interrogation tool is integrated with an RF transceiver. In embodiments, the sensors are empowered and interrogated by the RF transceiver from a distance, for example a distance of greater than 10 m, or alternatively from the surface or from an adjacent offset well. In an embodiment, the data interrogation tool traverses within a casing in the well and reads sensors located in a wellbore servicing fluid or composition, for example a sealant (e.g., cement) sheath surrounding the casing, located in the annular space between the casing and the wellbore wall. In embodiments, the interrogator senses the sensors when in close proximity with the sensors, typically via traversing a removable downhole component along a length of the wellbore comprising the sensors. In an embodiment, close proximity comprises a radial distance from a point within the casing to a planar point within an annular space between the casing and the wellbore. In embodiments, close proximity comprises a distance of 0.1 m to 1 m. Alternatively, close proximity comprises a distance of 1 m to 5 m. Alternatively, close proximity comprises a distance of from 5 m to 10 m. In embodiments, the transceiver interrogates the sensor with RF energy at 125 kHz and close proximity comprises 0.1 m to 5 m. Alternatively, the transceiver interrogates the sensor with RF energy at 13.5 MHz and close proximity comprises 0.05 m to 0.5 m. Alternatively, the transceiver interrogates the sensor with RF energy at 915 MHz and close proximity comprises 0.03 m to 0.1 m. Alternatively, the transceiver interrogates the sensor with RF energy at 2.4 GHz and close proximity comprises 0.01 m to 0.05 m.

In embodiments, the sensors are incorporated into wellbore cement and used to collect data during and/or after cementing the wellbore. The data collection component may be positioned downhole prior to and/or during cementing, for example integrated into a component such as casing, casing attachment, plug, cement shoe, or expanding device. Alternatively, the data collection component is positioned downhole upon completion of cementing, for example conveyed downhole via wireline. The cementing methods disclosed herein may optionally comprise the step of foaming the cement composition using a gas such as nitrogen or air. The foamed cement compositions may comprise a foaming surfactant and optionally a foaming stabilizer. The MEMS sensors may be incorporated into a sealant composition and placed downhole, for example during primary cementing (e.g., conventional or reverse circulation cementing), secondary cementing (e.g., squeeze cementing), or other sealing operation (e.g., behind an expandable casing).

In primary cementing, cement is positioned in a wellbore to isolate an adjacent portion of the subterranean formation and provide support to an adjacent conduit (e.g., casing). The cement forms a barrier that prevents fluids (e.g., water or hydrocarbons) in the subterranean formation from migrating into adjacent zones or other subterranean formations. In embodiments, the wellbore in which the cement is positioned belongs to a horizontal or multilateral wellbore configuration. It is to be understood that a multilateral wellbore configuration includes at least two principal wellbores connected by one or more ancillary wellbores.

### NEMS-MEMS Sensor Power Source

In embodiments, the data sensors added to the wellbore composition, e.g., cement, etc., are passive sensors that do not require continuous power from a battery or an external source in order to transmit real-time data. In embodiments, the data sensors are NEMS or MEMS comprising one or more and typically a plurality of NEMS/MEMS devices, referred to herein as NEMS or MEMS sensors. NEMS/MEMS devices are well known, e.g., a semiconductor device with mechanical features on the micrometer scale. NEMS/MEMS embody the integration of mechanical elements, sensors, actuators, and electronics on a common substrate. in embodiments, the substrate comprises silicon. NEMS/MEMS elements include mechanical elements which are movable by an input energy (electrical energy or other type of energy). Using NEMS/MEMS, a sensor may be designed to emit a detectable signal based on a number of physical phenomena, including thermal, biological, optical, chemical, and magnetic effects or stimulation. NEMS/MEMS devices are minute in size, have low power requirements, are relatively inexpensive and are rugged, and thus are well suited for use in wellbore servicing operations.

In embodiments, the NEMS/MEMS sensors added to a cement may be active sensors, for example powered by an internal battery that is rechargeable or otherwise powered and/or recharged by other downhole power sources such as heat capture/transfer and/or fluid flow, as described in more detail herein.

In certain embodiments, dielectric materials, that respond in a predictable and stable manner to changes in parameters over a long period may be identified according to methods well known in the art, for example see, e.g., Ong, Zeng, and Grimes. “A Wireless, Passive Carbon Nanotube-based Gas Sensor,” IEEE Sensors Journal, 2, 2, (2002) 82-88; Ong, Grimes, Robbins and Singl, “Design and application of a wireless, passive, resonant-circuit environmental monitoring sensor,” Sensors and Actuators A, 93 (2001) 33-43, each of which is incorporated by reference herein in its entirety. Sensors suitable for the methods of the present disclosure that respond to various wellbore parameters are disclosed in U.S. Pat. No. 7,038,470 B1 that is incorporated herein by reference in its entirety.

In other embodiments, the NEMS-MEMS sensors include a radio frequency identification devices (RFIDs) and can thus detect and transmit parameters and/or well cement characteristic data for monitoring the cement during its service life. In certain embodiments, the RFIDs include power when exposed to a narrow band, high frequency electromagnetic field from a transceiver. A dipole antenna or a coil, depending on the operating frequency, connected to the RFID chip, powers the transponder when current is induced in the antenna by an RF signal from the transceiver's antenna. Such a device can return a unique identification number by modulating and re-radiating the radio frequency (RF) wave. in certain embodiments, passive RF tags include low cost, indefinite life, simplicity, efficiency, ability to identify parts at a distance without contact (tether-free information transmission ability). These robust and tiny tags are attractive from an environmental standpoint as they require no battery. The NEMS- or MEMS sensor and RFID tag are preferably integrated into a single component or may alternatively be separate components operably coupled to each other. In an embodiment, an integrated, passive NEMS- or MEMS RFID sensor contains a data sensing component, an optional memory, and an RFID antenna, whereby excitation energy is received and powers up the sensor, thereby sensing a present condition and/or accessing one or more stored sensed conditions from memory and transmitting same via the RFID antenna.

In embodiments, NEMS- or MEMS sensors having different RFID tags, i.e., antennas that respond to RF waves of different frequencies and power the RFID chip in response to exposure to RF waves of different frequencies, may be added to different wellbore.

Piezoelectric sensors can scavenge acoustic power and store it in local energy storage devices (e.g. capacitors, supercapacitors, batteries) The integrated, inexpensive sensors remain in place as a set of distributed sensors to enable robust data collection over time. These swarms of sensors comprise multiple distinct units that behave similarly and collectively enable the gathering and transmission of data about their location and environment. The sensors emit a return pulse in response to acoustic or electromagnetic queries by retrievable transducers. Affordable mass manufacturing of integrated smart sensors presents a good opportunity to build smart cement elements.

Smart cement sensors respond to acoustic or radio frequency queries by emitting a return pulse. The location of these pulses enables the location of the sensors to be identified. Understanding these locations enables three-dimensional tomographic mapping and characterization of the cement locations. In addition to identifying cement coverage, the smart sensors could potentially measure the local temperature, pressure, stress/strain relationships, micro-acoustic fracturing, flowing fluids, pH, presence and concentration of particular ions, humidity, vibrations and other parameters such as those related to microporosity and the structural environment including rock types, bedding structures and the borehole/well-casing environment.

In certain embodiments, the NEMS- or MEMs sensors include a slurry of, for example, millions of micron-scale sensors that provide data wirelessly to an instrument hub or hub arrays, with the hub or hub arrays relaying data to the back-end processing system. Remote sensors can collect a variety of environmental data in large amounts, including temperature, pressure, salinity, pH, vibration, shear stress and strain, acoustic signature and flow data.

In one embodiment, the remote sensor contains a piezoelectric transducer capable of harvesting power from the hub through the rock into the sensor by retrieving energy from shear and/or compression waves (S waves and P waves respectively). The sensor may respond in a ping or transponder mode by sending an “I am here” response and providing location information through acoustic transponding. The sensor may also respond in a data mode by collecting lots of data over time and transferring the data in bursts or packets. Smart sensors may also include energy storage capability for permanent monitoring over long periods of time.

Wireless power transmission can be enabled using radio frequency (RF) transmission from the wellhead to the hubs (nodes) and acoustic transmission to and from the sensors. Wireless acoustic power transmission inside the casing is also a possibility, EM power transmission on a kilowatt (KW) scale down a wellbore in combination with acoustic power conversion on a watt scale by hubs achieves wireless power transmission. Hubs (nodes) can be deployed inside or outside the casing. There may additionally be a cementing dielectric sheath around the casing.

The sensors may form a network using wireless links to neighboring data sensors and have location and positioning capability through, for example, local positioning algorithms as are known in the art. The sensors may organize themselves into a network by listening to one another, therefore allowing communication of signals from the farthest sensors towards the sensors closest to the interrogator to allow uninterrupted transmission and capture of data. In such embodiments, the hub may not need to traverse the entire section of the wellbore containing NEMS/MEMS sensors in order to read data gathered by such sensors, For example, the hub may only need to be lowered about half-way along the vertical length of the wellbore containing sensors. Alternatively, the hub may be lowered vertically within the wellbore to a location adjacent to a horizontal arm of a well, whereby sensors located in the horizontal arm may be read without the need for the hub to traverse the horizontal arm. Alternatively, the hub may be used at or near the surface and read the data gathered by the sensors distributed along all or a portion of the wellbore. For example, sensors located a distance away from the hub (e.g., at an opposite end of a length of casing or tubing) may communicate via a network formed by the sensors as described previously.

1. A sensor component comprising:
i. a temperature sensing element;
ii. a pressure sensing element;
iii. a stress/strain sensing element; and
iv. an acoustic sensing element
wherein the sensor component is on the scale of about centimeters to about microns.

2. A cement monitoring composition comprising a plurality of wireless sensors, wherein each sensor comprises:
a. a sensor component comprising:
i. a temperature sensing element;
ii. a pressure sensing element;
iii. a stress/strain sensing element; and
iv. an acoustic sensing element
wherein the sensor component is on the scale of centimeters to about microns.

3. The cement monitoring composition of claim 2, wherein the sensor component comprises a polymer material.

4. The cement monitoring composition of claim 3, wherein the polymer material comprises a polymer film material.

5. The cement monitoring composition of claim 4, wherein the polymer film material comprises polyimide.

6. The cement monitoring composition of claim 2, wherein the sensor component comprises a ceramic material.

7. The cement monitoring composition of claim 6, wherein the ceramic material comprises a ceramic perovskite material.

8. The cement monitoring composition of claim 6, wherein the ceramic material is lead zirconium titanate.

9. The cement monitoring composition of claim 2, wherein the sensor component has a dielectric constant from about 200 to about 4000.

10. The cement monitoring composition of claim 2, wherein the sensor component comprises a piezoelectric material.

11. The cement monitoring composition of claim 2, wherein the temperature sensing element is a temperature diode.

12. The cement monitoring composition of claim 2, wherein the temperature sensing element is a thermistor.

13. The cement monitoring composition of claim 2, wherein the pressure sensing element is a pressure sensitive ink.

14. The cement monitoring composition of claim 2, wherein the pressure sensing element is a pressure sensitive transducer.

15. The cement monitoring composition of claim 2, wherein the pressure sensing element comprises a passivation layer.

16. The cement monitoring composition of claim 2, wherein the stress/strain sensing element is a nanoparticle-based strain gauge.

17. The cement monitoring composition of claim 2, wherein the stress/strain sensing element is a foil strain gauge.

18. The cement monitoring composition of claim 2, wherein the stress/strain sensing element comprises an interdigitated transducer.

19. The cement monitoring composition of claim 2, further comprising one or more data collection components.

20-24. (canceled)

25. A method of monitoring a cement comprising:
a. providing a plurality of wireless sensors in a cement, wherein each sensor comprises:
i. a sensor component comprising:
1. a temperature sensing element;
2. a pressure sensing element;
3. a stress/strain sensing element; and
4. an acoustic sensing element
b. adding the cement to a wellbore;
c. obtaining data from the sensors using a plurality of data collection components spaced along a length of the wellbore; and
d. transmitting the data obtained from the sensors from an interior of the wellbore to an exterior of the wellbore.

26-47. (canceled)
